(19) United States
(12) Patent Application Publication
(10) Pub. No.: US 2004/0012758 A1
(43) Pub. Date: Jan. 22, 2004

(54) Prism based dynamic vision training device and method thereof

(76) Inventor: Chao-Chyun Lin, Taipei (TW)

Correspondence Address: Jason Z. LIN, Supreme Patent Services, Post Office Box 2339, Saratoga, CA, 95070-0339, US

(21) Appl. No.: 10/456,392

(22) Filed: Jun. 5, 2003

(30) Foreign Application Priority Data
Jul. 19, 2002 (TW) 91116074
Apr. 21, 2003 (TW) 92109252

(51) Int. Cl. G02C 7/02; A61B 3/00

(52) U.S. Cl. 351/203

(57) ABSTRACT

A vision training device includes a fixed frame positionable in front of a wearer's face. The fixed frame defines two windows corresponding in position to the eyes of the wearer, through which light passes. An optic system includes a prism lens, which may have fixed power or variable power by changing shapes thereof. The prism lens is mounted to the fixed frame and is movable between first and second positions, wherein in the first position, light is allowed to pass in a first state with which the eyes are adducted, and in the second position, light is allowed to pass in a second state with which the eyes are abducted. A transmission system is coupled to and selectively drives the prism lens between the first and second positions. Thus, by repeatedly and cyclically moving the prism lens between the first and second positions, the eyes are forced to change between adduction and abduction thereby realizing training of vision.

### BACKGROUND OF THE INVENTION

1. Field of the Invention

The present invention relates generally to a method for training and thus improving vision of human beings, especially the nearsighted, and in particular to repeatedly and cyclically moving prisms in front of and away from eyes to forcibly abduct eyeballs in order to exercise and relax eyeball movement muscles thereby slowing down potential myopic progress and decreasing severity of myopia.

2. The Related Art

The structure of a human eye is similar to a camera. Generally, a human eye has ciliary muscles controlling the thickness of the lens, and thus causing accommodation of the lens to form a clear image when the eye looks at an object located at either a short distance or a far distance. Six extraocular muscles act on the eyeball and control the movement of the eye. The extraocular muscles of the two eyeballs of an individual coordinate together to look towards the same direction and focus on the same object. When looking at a near distance, the two eyeballs adduct to focus on the same object. When the object is at a far distance, on the contrary, the two eyeballs abduct.

It is a known fact of ophthalmology that the adduction and abduction of the eyeballs, which cause convergence and divergence of the eyeballs respectively, work synergistically with accommodation of the lenses to enhance the process of focusing. When focusing at a close-distanced object, the eyeballs adduct, aiding the contraction of the ciliary muscles, which thickens the lenses to form a clear image. This is referred to as “accommodation”. On the other hand, when focusing at a far-distanced object, the abduction of the two eyeballs helps in relaxing the ciliary muscles to slim up the lenses, hence forming a clear image for the far-distanced object. This is referred to as “relaxation of accommodation”.

In the past decades, due to modernization and changes in lifestyle, there has been a dramatic increase in the need for individuals to sustain constant short-range viewing. Long durations of short-range work, such as writing, reading, operating computers and watching television, require prolonged contraction of the ciliary muscles and the internal rectus muscles, making the muscles stiffened. This is especially likely in young people, whose eyes are still in development. Due to the stiffened ciliary muscles, the thickened lenses are difficult or even unable to become thin again when viewing distant objects. The image of the distant object thus falls in front of the retina and becomes unclear, thus causing myopia.

There are two types of myopia: “functional myopia” (refractive myopia) and “structural myopia” (axial myopia), differentiated by their mechanism of formation. Functional myopia is formed by over-contraction of the ciliary muscles, which causes over-thickening of the lenses, making image of a distant object fall in front of the retina. In “structural myopia”, the lenses are normal, but the oculi axes are too long to make the image fall in front of the retina.

All myopias begin with functional myopia, including the so called “pseudo-myopia”. In the functional myopia, if the eye movement muscles (extraocular and intraocular muscles) are unable to relax due to prolonged hours of constant short-range viewing, the eyeballs start to adapt to the situation by increasing the length of the oculi axis so that the image of close objects fall on the retina, inducing formation of structural myopia. This acquired myopia can be found in most modernized countries.

The progression of myopia is the result of a vicious circle of the functional myopia and the structural myopia. Therefore, if the functional myopia can be controlled and over-lengthening of the oculi axes can be prevented, the progression of the structural myopia can be stopped or at least slowed down.

Humanoid has their eyes side by side in front of the head and is, by default, accustomed to convergence rather than divergence. Due to the arrangement of the eyes, the most abducted eye position is usually that of a parallel vision occurring when viewing a far-distanced object. Theoretically, increasing abduction of the eyeballs to an eye position that is more abducted than that of a parallel vision will balance out over-adduction that comes along with the modern life-style.

The ophthalmological facts are as follows. With increased adduction, accommodation of eye increases. On the other hand, when the eyes abduct, accommodation decreases, namely relaxation of accommodation. Therefore, it can be said that, prolonged duration of adduction and accommodation is the cause of myopia and its progression.

Long hours of viewing with the eyes, compounded by a fixed focal length, especially a short fixed focal length, is the most common cause of myopia. People with normal vision (namely, a person having no myopia) is able to have clear images of both close and far distanced objects because their eye movement muscles (extraocular and intraocular muscles) remain agile and not stiffened due to less time of short-distance fixed focal length.

Muscles, such as those of legs, arms and the rest of human body, start aching and get stiffened when the muscles fixed in one position for long while. However, periodical exercise maintains the muscles agile and prevent the muscles from getting aching. The eye movement muscles (extraocular and intraocular muscles) are of no exception. Constant change of focal length by movement of the eyeballs effectively prevents the eye muscles from stiffening, thus preventing myopia. Therefore, by maintaining constant movement of the eye muscles, especially within short durations of time, during short-range viewing, myopia can be prevented and giving up short-range viewing in an effort to prevent myopia is unnecessary. That is, the eyeballs must change positions among adduction, abduction, accommodation, and relax of accommodation, in a short time for protection purposes. The focal length is altered constantly to prevent myopia from occurring, as myopia is caused by long hours of looking with a short fixed focal length.

A number of vision training devices are available in the market. These known vision training device all emphasize on exercising eyeballs. Some of these devices train the extraocular muscles by having eyes follow a series of lights, while the others train the intraocular muscles by having eyes look at one object that constantly moves toward and away from the viewer. These known devices are only good in simultaneously moving the two eyeballs as a whole and are not able to affect abduction of the eyeballs. The training result of these known devices is in general not very good, because the “optimal relaxation of accommodation” can only be achieved by the abduction of the two eyeballs and the use of convex lenses that substitute for the contraction of the ciliary muscles.

Clinically, the lengthening speed of the oculi axes in structural myopia is far greater than that of the normal growth lengthening. Hence, with an ophthalmoscope, a bluish crescent can be found at the temporal side of the optic disk on the retina, which is called the temporal choroidal crescent or more commonly, the myopic crescent. A possible explanation of this phenomenon is that the eyeballs are, by functional requirement, constantly held in adduction for long durations. The optic nerve is situated at the posterior of the eyeball closer to the nasal side. When the eyeballs adduct, the corneal section turns towards the nasal side, while the posterior section of the eye turns towards the temporal side, causing the temporal side of the junction of the optic nerve and the eyeball to be stretched, forming the myopic crescent, and lengthening the posterior wall. Therefore, adduction of the eyeballs could be the reason for lengthening of the oculi axes and the formation of the myopic crescent.

Further, the conventional vision training devices require a predetermined and devoted period of time every day for using the device and training the eyes. People often find that it is troublesome and boring to take the training by using the device everyday. Thus, most people are not able to continue with the training sessions day after day, let alone for months or years. Training is thus often abandoned shortly after commencement. As a result, these conventional devices are considered ineffective.

Thus, the present invention is aimed to provide a dynamic vision training device that overcomes the deficiencies of the prior art and effectively improve human vision by slowing down the speed of myopic progression and alleviating myopia.

### SUMMARY OF THE INVENTION

An object of the present invention is to provide a vision training method comprising constantly placing and removing prism lenses in front of a viewer's eyes to separate one single viewing object into double images. The uncomfortable double vision is immediately processed by an image fusion mechanism of the viewer's brain, which causes the eyeballs to abduct for eliminating the double images. The hardest one of the eyeball movements, namely the abduction, can thus be achieved. The abduction of the eyes helps relaxing the overly contracted internal rectus muscles and is also helpful in relaxing accommodation of lenses of the eyes.

Another object of the present invention is to provide a vision training method comprising constantly placing and removing convex prism lenses comprising a prism portion integrally formed on or additionally mounted to a convex lens in front of a viewer's eyes whereby, during the abduction that helps relaxing accommodation, the plus power of the convex lens substitutes accommodation of the eyes in short-range viewing.

A further object of the present invention is to provide a vision training method, which coordinates the time of use of convex and prism lenses for vision training in order to relax the internal rectus muscles, achieve constant change of focal length and induce total relaxation of accommodation.

A further object of the present invention is to provide a vision training method that continuously exercises eye movement muscles, including extraocular and intraocular muscles, in a short period, such as few seconds and up to tens of seconds, to relax the eyeballs and prevent myopic progression.

A further object of the present invention is to provide a vision training method comprising using prism lenses to reduce adduction and thus prevent structural myopia and increase abduction of eyeballs. The abduction forces optic nerve and the posterior section of the eyeball in the opposite direction to that of adduction and hence reduces the effect of structural myopia.

A further object of the present invention is to provide a vision training device that can be used during normal “working” time The device can be worn when a wearer is writing, operating computers, and even watching television to relax eye movement muscles of the wearer unwittingly. The daily life of the wearer is in general not affected and the wearer does not feel the training process troublesome or boring.

A further object of the present invention is to provide a vision training method comprising placing prism lenses and/or convex lenses in front of eyes of a person wherein the placement of the prism lenses in front of the eyes is dynamically set so that the length of time, such as 10-30 seconds, during which the prism lenses and the convex lenses are placed in front of the eyes is longer than the time, such as 5-20 seconds, during which the prism lenses or the convex lenses are removed in order to allow the eye movement muscles, including extraocular and intraocular muscles, to achieve the effect of eyeball abduction and relaxation of accommodation by giving the eyes shorter time for adduction and accommodation.

A further object of the present invention is to provide a vision training device that is designed to wear on the head, or positioned over the eyes as glasses or eyeshade, or a tabletop type device.

A further object of the present invention is to provide a vision training device in which a combination of convex, concave and prism lenses is selected to coordinate with the different viewing distances of different users and to substitute the glasses of the myopic users.

To achieve the above objects, in accordance with the present invention, there is provided a vision training device comprising a fixed frame positionable in front of a wearer's face. The fixed frame defines two windows corresponding in position to the eyes of the wearer, through which light passes. An optic system comprises a prism lens, which may have fixed power or variable power by changing shapes thereof. The prism lens is mounted to the fixed frame and is movable between first and second positions, wherein in the first position, light is allowed to pass in a first state with which the eyes are adducted, and in the second position, light is allowed to pass in a second state with which the eyes are abducted. A transmission system is coupled to and selectively drives the prism lens between the first and second positions. Thus, by repeatedly and cyclically moving the prism lens between the first and second positions, the eyes are forced to change between adduction and abduction thereby realizing training of vision.

### BRIEF DESCRIPTION OF THE DRAWINGS

The present invention will be apparent to those skilled in the art by reading the following description of preferred embodiments thereof, with reference to the attached drawings, in which:

FIG. 1 is a schematic view showing the eyesight of a person looking at a short distance object whereby the eyeballs are adducted;

FIG. 2 is a schematic view showing the principle of the present invention wherein prism lens are placed in front of the eyes of a person to make the eyeball abducted when the person is looking at a short distance object;

FIG. 3 is a schematic view showing a conventional convex lens;

FIG. 4 is a schematic view showing a convex-prism lens to be incorporated in a vision training device constructed in accordance with the present invention;

FIG. 5 is a side elevational view showing the vision training device of the present invention worn on the head of a wearer;

FIG. 6 is a cross-sectional view of a vision training device in accordance with a first embodiment of the present invention observed from a top side thereof;

FIG. 7 is a front view of the vision training device of the first embodiment of the present invention;

FIG. 8 is a cross-sectional view of a vision training device in accordance with a second embodiment of the present invention observed from a top side thereof,

FIG. 9 is a front view of the vision training device of the second embodiment of the present invention;

FIG. 10 is a front view similar to FIG. 9 but showing prism lens of the vision training device in non-operating positions;

FIG. 11 is a cross-sectional view of a vision training device in accordance with a third embodiment of the present invention observed from a top side thereof, some components being removed for simplicity;

FIG. 12 is a front view of the vision training device of the third embodiment of the present invention;

FIG. 13 is a front view similar to FIG. 12 but showing prism lens of the vision training device in non-operating positions;

FIG. 14 is a side elevational view showing a vision training device constructed in accordance with a fourth embodiment of the present invention worn on the head of a wearer;

FIG. 15 is a front view of the vision training device of the fourth embodiment of the present invention;

FIG. 16 is a front view of a vision training device in accordance with a fifth embodiment of the present invention;

FIG. 17 is a front view of a vision training device of the fifth embodiment of the present invention but showing prism lens in non-operating position;

FIG. 18 is a front view of a vision training device constructed in accordance with a sixth embodiment of the present invention;

FIG. 19 is a front view of the vision training device of the sixth embodiment of the present invention but showing prism lens in non-operating position;

FIG. 20 is a side elevational view of a vision training device constructed in accordance with a seventh embodiment of the present invention;

FIG. 21 is a cross-sectional view of a vision training device constructed in accordance with an eighth embodiment of the present invention, observed from a front side thereof;

FIG. 22 is a cross-sectional view of the vision training device of the eighth embodiment of the present invention observed from a top side thereof;

FIG. 23 is a cross-sectional view of a vision training device constructed in accordance with a ninth embodiment of the present invention, observed from a front side thereof;

FIG. 24 is a cross-sectional view of a vision training device constructed in accordance with a tenth embodiment of the present invention, observed from a front side thereof;

FIG. 25 is a cross-sectional view of the vision training device of the tenth embodiment of the present invention observed from a top side thereof;

FIG. 26 is a cross-sectional view of a vision training device constructed in accordance with an eleventh embodiment of the present invention, observed from a front side thereof.

### DETAILED DESCRIPTION OF THE PREFERRED EMBODIMENTS

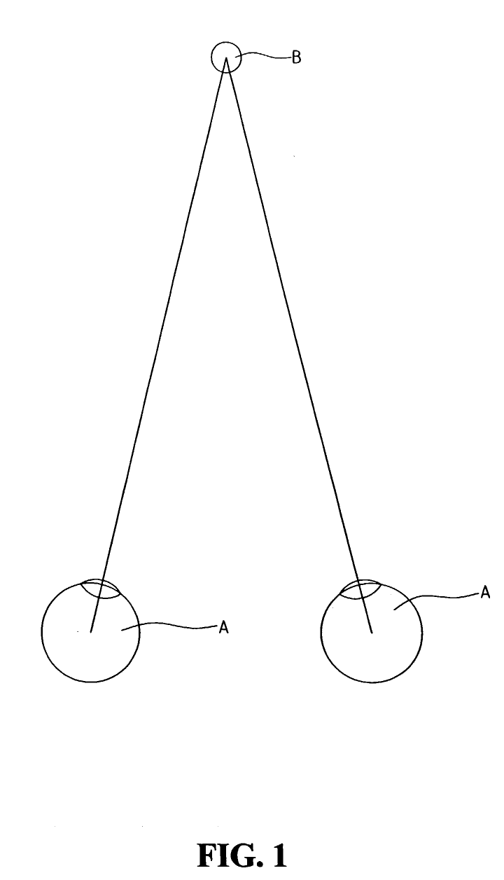

With reference to the drawings and in particular to FIGS. 1-4, a description of the principle of vision training in accordance with the present invention will be given first before details of the constructions of preferred embodiment are illustrated. FIG. 1 shows a general condition when a person has his or her eyeballs A looking at an object B, which is located nearby. Due to the image fusion mechanism of human brain, the eyeballs A are adducted to form a single image. In order to realize abduction of the eyeballs A, in accordance with the present invention, a prism lens C is placed in front of each eyeball A whereby the eyeballs A are abducted, again, to avoid formation of double images.

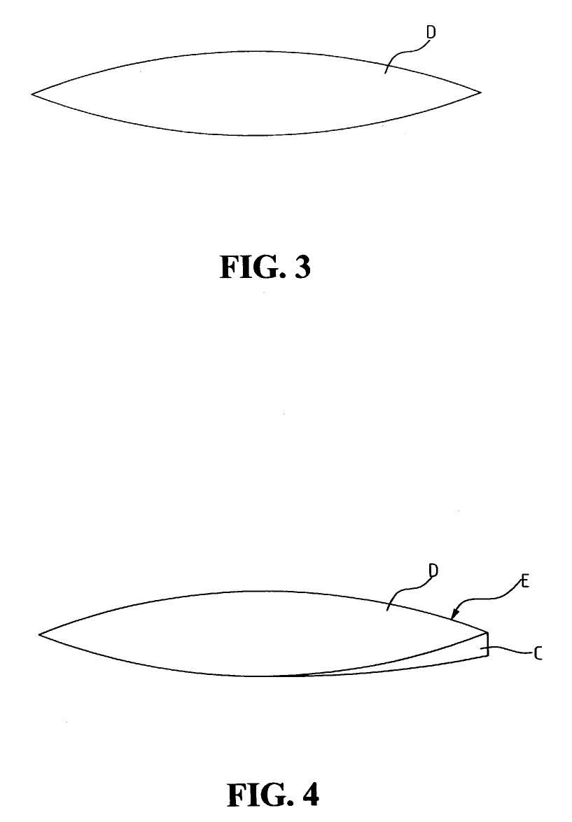

In accordance with the present invention, a prism lens may be separately used or used in combination with a convex lens. FIG. 3 shows a conventional convex lens D. A prism lens C can be integrated with the convex lens D to form a convex-prism lens E in accordance with the present invention. The convex-prism E provides vision training in accordance with the present invention while at the same time allows for a nearsighted wearer to perform his or her daily job without being subject to undesired constraints that is commonly observed in the conventional vision training devices. One way to make the convex-prism lens E is to grind a prism lens C to form a convex configuration on one or two sides thereof. This is a well-known technique is the field of lens-making and thus no further details will be given herein. It is noted that in the following description, the term “prism lens” may sometimes include the “convex-prism lens”.

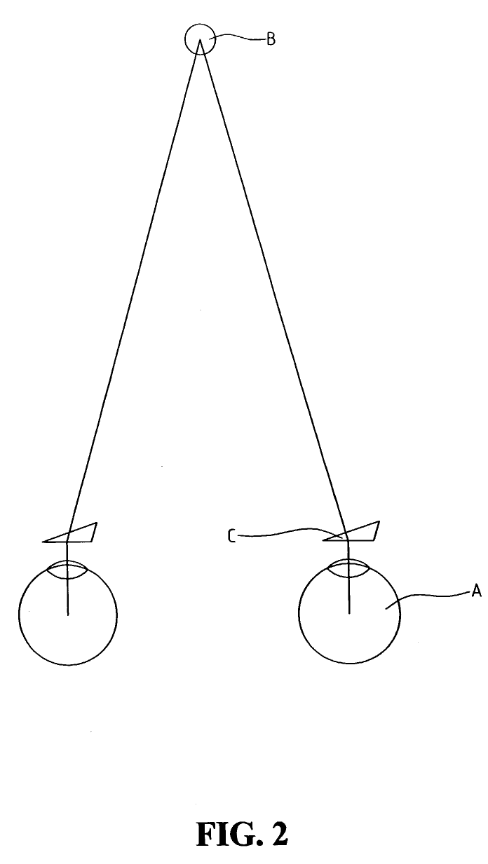

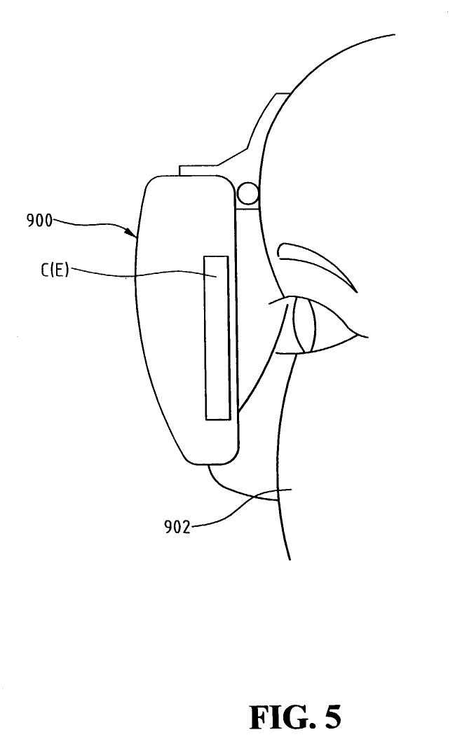

Referring to FIG. 5, a vision training device constructed in accordance with the present invention is broadly designated with reference numeral 900 is positioned in front of a wearer's head 902. The vision training device 900 comprises prism lens C or convex-prism lens E aligned with the eyeballs A of the wear 902 for changing the eyesight from a non-abducted condition to an abducted condition as respectively illustrated in FIGS. 1 and 2. With this, the eyeballs A are forced to abduct and thus achieving vision training in accordance with the present invention.

In the following description, for purpose of simplification, the prism lens C or the convex-prism lens E has a thicker side which will be referred to as “base” of the lens and is used as a benchmark. The lens C (or E) are referred to as “base in” when two lens C (E) are positioned in front of the eyeballs A of the wearer 902 with the bases thereof close to each other wherein the eyeballs A abduct as illustrated in FIG. 2. In other words, the bases of the lens are facing each other. On the other hand, when the bases of the two lenses are oriented away from each other in opposite directions, it is referred to as “base out”. When the bases point downwards at the time when the vision training device 900 is worm, it is referred to as “base down” wherein the eyeballs A turn upwards.

As human life becomes more and more civilized in recent years when compared to that of the old time when there have been fewer cases of myopia, there is an increase in short-range viewing, where the eyeball movement is generally inward and downward, but rarely upward. To correct the undesired consequence of the short-range viewing, the prism lens C or the convex-prism lens E are placed at the base-down and base-in positions to increase the upward-turning action and abduction of the eyeballs A, thus training the extraocular muscles to move smoothly in every direction and hence slowing down the speed of myopic progression.

In accordance with the present invention, when the wearer 902 looks at a distant object, the base-in and base-out positions of the prism lenses C (or the convex-prism E) interchange for the eyeballs A to repeatedly and cyclically abduct and adduct. Alternatively, the base-out condition of the prism lens C (or convex-prism lens E) can be replaced by no prism lens C and the operation of the vision training device 900 of the present invention becomes repeated and cyclical base-in and “no prism lenses”.

The length of time that the wearer has to see through the prism lenses for vision training purposes is dependent on whether the wearer is doing short-range or long-range viewing. For example, in a short-range viewing (writing, reading or operating computers), the time period with the prism lens C or the convex prism lens E (that is base-in) is about 20 seconds and the time period without the prism lens C or the convex-prism lens E (that is base-out) is about 6 seconds. In a long-range viewing, such as watching TV, the time period with the prism lens C or the convex-prism lens E (that is base-in) is about 10 seconds and the time period without the prism lens C or the convex-prism lenses E (that is base-out) is about 6 seconds.

In general, the time period with the prism lens C or the convex-prism lens E on is approximately between 10-30 seconds. If the time period is less than 10 seconds, dizziness can result, due to the rapid change. If the time period is greater than 30 seconds, the training result is reduced.

The time period with the lens C or E off or at the base-out position is approximately between 5-20 seconds. During this time, the eye movement muscles, such as the extraocular and intraocular muscles, return to their contracted and tense state. Thus the time period for base-out or “no prism lens” should not be too long, so as to reach the intended purpose of this invention for exercising and relaxing the eyeballs.

In accordance with the present invention, the change of the prism lens or the convex-prism lens includes change of degree of power and change of position. In the description, it is assumed same degree of power for the prism lens and/or the convex power for both eyes. The “degree of power” mentioned hereafter is for that of one single eye. During short-range viewing, the degree of power used should be greater than the degree used at long-range viewing. This is because the degree of adduction at short-range viewing is more than the degree of adduction at long-range viewing. Hence a greater prism power is needed for the eyeballs to abduct.

Thus, the degree of prism power and convex power for short-range viewing is:

Prism power: 4 ΔDiopter−10 ΔDiopter (for single eye)

Convex power: +1.0 Diopter−+3.0 Diopter (for single eye)

During long-range viewing, the degree of power used should be less than the degree used at short-range viewing. The degree of adduction is already small during long-range viewing, so if a too powerful prism degree is used, double vision will occur due to the brain being unable to perform the image fusion mechanism. If the prism power is less than 3 ΔDiopter, the degree of eyeball abduction caused is too small, and the training result is limited. However, if the prism power is too great, double vision will occur and it is not possible to perform the image fusion mechanism.

Thus the degree of prism power and convex power for long-range viewing is:

Prism power: 3 ΔDiopter−8 ΔDiopter (for single eye)

Convex power: +0.25 Diopter−+0.75 Diopter (for single eye)

Clinical trials show that the degree of prism power used should differ from person to person. People with exophoria have a more abducted eye position normally and thus a greater degree of prism power can be used. People with esophoria, on the other hand, should use prism lenses that are less powerful. This person-to-person differences of the prism power needed can be overcome by changing the prism lens.

The degree of the convex lens power is approximately between +0.25 Diopter−+3.0 Diopter. This includes the total convex power used. In the present invention, the described degree of convex power is only for the single eye and if the prism lenses are superimposed, the degree of power described should then be the total of all lenses used for one single eye.

During short-range viewing, the degree of convex lens power should be more than the degree used during long-range viewing. The degree of convex power should be inversely proportionate to the “distance to the object” viewing, that is, the further the object is, the lesser the power should be used. The actual use of convex power in this invention should be +0.25 Diopter−+0.75 Diopter more than the convex power calculated optically, so as to produce a fogged vision, which facilitates the total relax of accommodation.

For example if a person is using the vision training device 900 of the present invention with a viewing distance of 50 cm: 100 cm÷50 cm=2.0 Diopter. Then, the convex power used should be approximately +2.25 Diopter−+2.75 Diopter.

If a person is using the vision training device 900 of the present invention with a viewing distance of 33 cm: 100 cm÷33 cm=3.0 Diopter. Then, the convex power used should be approximately +3.25 Diopter−+3.75 Diopter.

In the present invention, the vision training device 900 can be made in a plurality of configurations. For example, it can also be designed to wear on the head 902 as shown in FIG. 5, wear over the eyes as glasses or eyeshade, or as a tabletop type device.

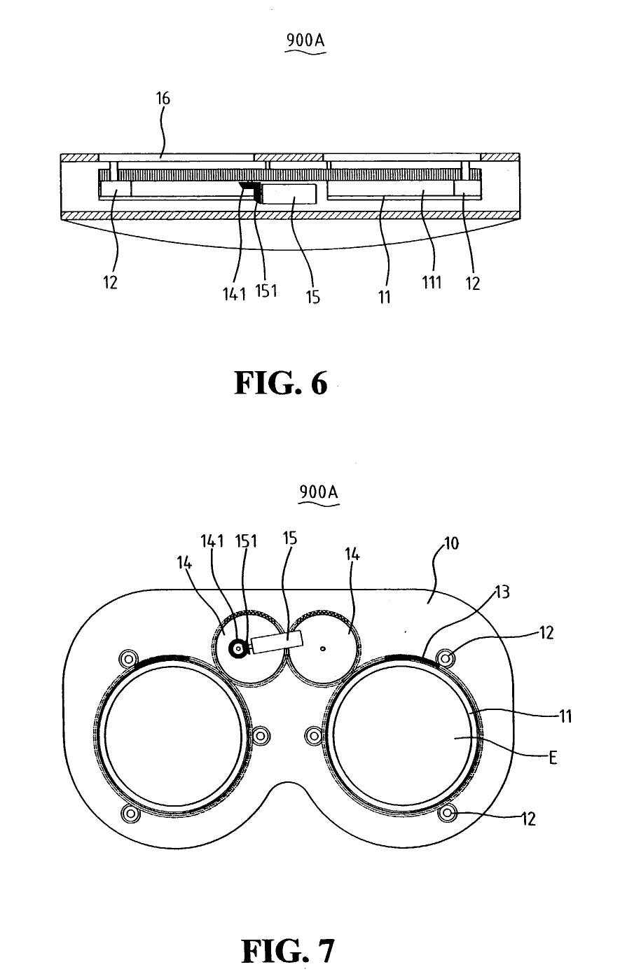

Also referring to FIGS. 6 and 7, the vision training device constructed in accordance with a first embodiment of the present invention is illustrated and is designated with reference numeral 900A for distinction. The vision training device 900A comprising a frame 10 defining two viewing windows 16 corresponding to the eyeballs A of the wearer 902. A plurality of rollers 12 is rotatably mounted to the frame 10 around each viewing window 16 along a circular trace (not shown). A lens, which as illustrated in the drawings, is a convex-prism lens E, but as mentioned above can be replaced by a regular prism lens C with a convex lens (not shown) mounted to each viewing window, is positioned in alignment with each viewing window 16 and retained and supported by the rollers 12 whereby the lens E is allowed to rotate about the center of the circular trace and thus moved between base-in and base-out positions.

The lens E is received and mounted in a ring 11 that forms a circumferential groove 111 along an outer circumference of the ring 11. The rollers 12 are received in and in friction engagement with the groove 111. Teeth 13 are formed on the outer circumference of the ring 11 for mechanically coupling with a driving device, such as a step motor 15 via a gear 14. The gear 14 mates the teeth 13 of the ring 11. A bevel gear 141 is coaxially mounted to the gear 14 and mates an output bevel gear 151 mounted to the motor 15. A control circuit (not shown) is devised to control the operation of the motor 15, which in turn drives the gears 14 to drive the rings 11 for moving the lens E between base-in and base-out positions.

The control circuit is known to those having ordinary skills in the field of electrical control and thus no further detail is needed herein.

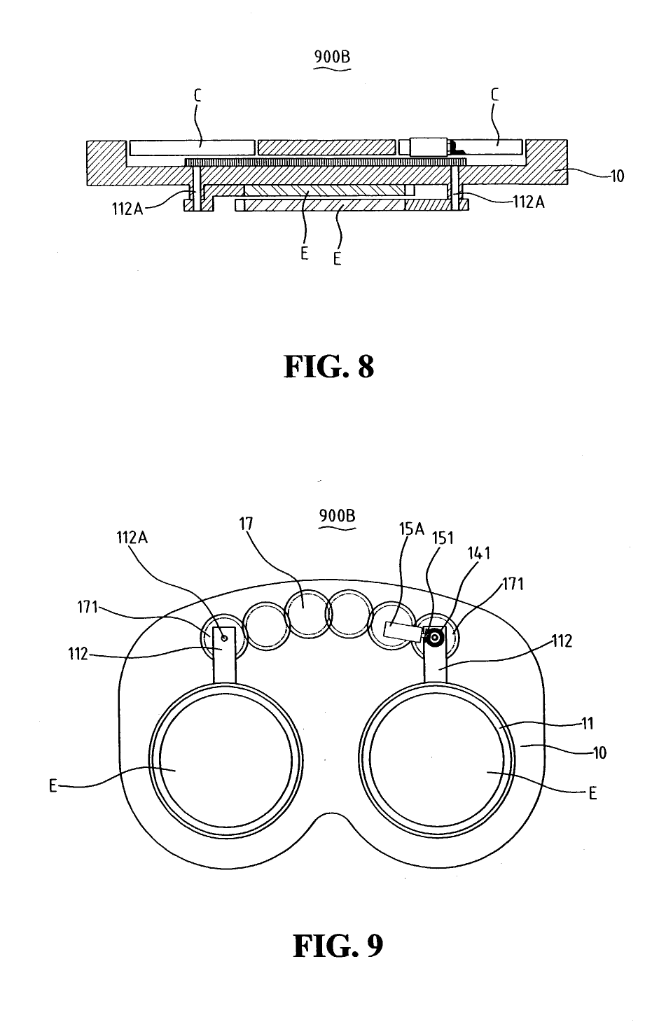

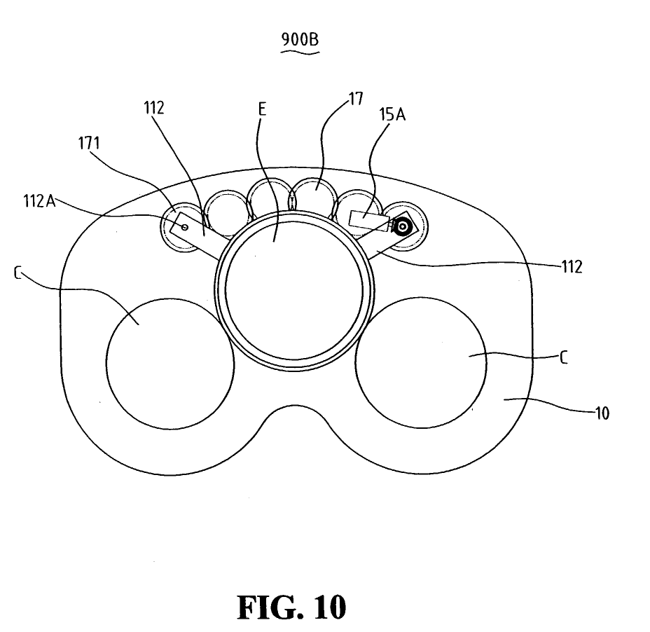

Also referring to FIGS. 8-10, the vision training device constructed in accordance with a second embodiment of the present invention is illustrated and designated with reference numeral 900B for distinction. The vision training device 900B comprises a fixed frame 10 defining two viewing windows (not labeled) in which two prism lens C are mounted. It is noted the prism lens C can be removed A swing arm 112 is rotatably mounted to the fixed frame 10 by a pivot shaft 112A and carries a ring 11 in which a convex-prism lens E is mounted. A pinion 171 is mounted to the swing arm 112 and is coaxial with the pivot shaft 112A. The pinion 171 is coupled to a drive device, such as a step motor 15A controlled by a control circuit (not shown), by means of a gear train comprised of at least one idle gear 17. By means of the operation of the step motor 15A, the swing arms 112 is rotated about the pivot shaft 112A and the lens E is moved between the base-in position in front of the viewing window as shown in FIG. 9 and the base-out position offset from the viewing window as shown in FIG. 10.

The motor 15A has an output bevel gear 151 that mates a driven bevel gear 141. The driven bevel gear 141 can be mounted to any one of the idle gears 17 or one of the swing arms 112 (or the associated pinion 171) as illustrated in the drawings.

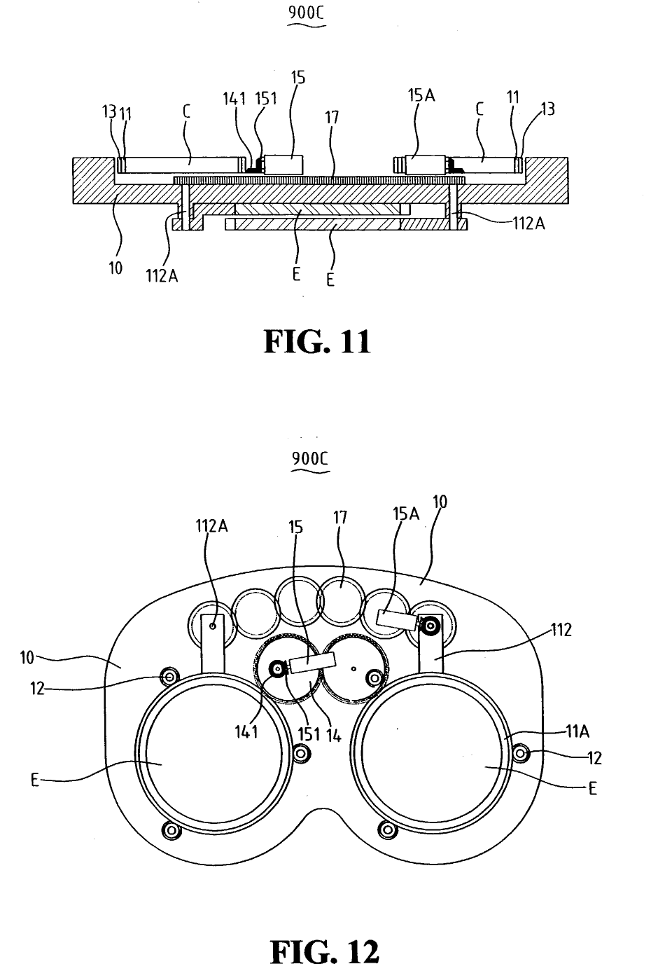

Referring to FIGS. 11-13, the vision training device constructed in accordance with a third embodiment of the present invention is illustrated and designated with reference numeral 900C for distinction. The vision training device 900C comprises a fixed frame 10 defining two viewing windows corresponding to which two prism lenses C that are retained in two first rings 11 are arranged. Each first ring 11 defines a circumferential groove along an outer circumference thereof for engaging rollers 12 that support the rotation of the first ring so as to move the prism lenses C between base-in and base-out positions. A first motor 15 controlled by a control circuit (not shown) is coupled to a gear 14 by a pair of bevel gears 151, 141 and the gear 14 mates external teeth (not labeled) formed on the circumference of the first ring 11.

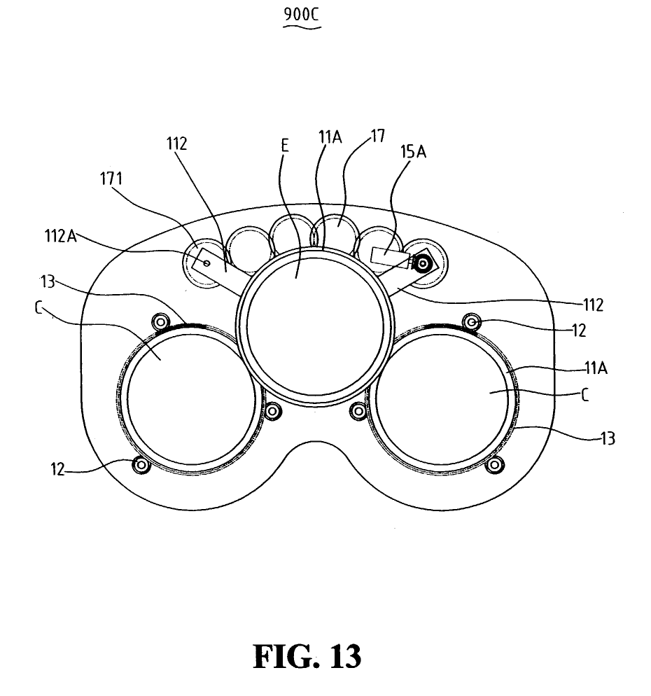

A swing arm 112 is rotatably mounted to the fixed frame 10 by a pivot shaft 112A and carries a second ring 11A in which a convex-prism lens E is mounted. A pinion 171 is mounted to the swing arm 112 and is coaxial with the pivot shaft 112A. The pinion 171 is coupled to a second motor 15A controlled by a control circuit (not shown), by means of a gear train comprised of at least one idle gear 17. By means of the operation of the step motor 15A, the swing arms 112 is rotated about the pivot shaft 112A and the lens E is moved between the base-in position in front of the viewing window as shown in FIG. 12 and the base-out position offset from the viewing window as shown in FIG. 13.

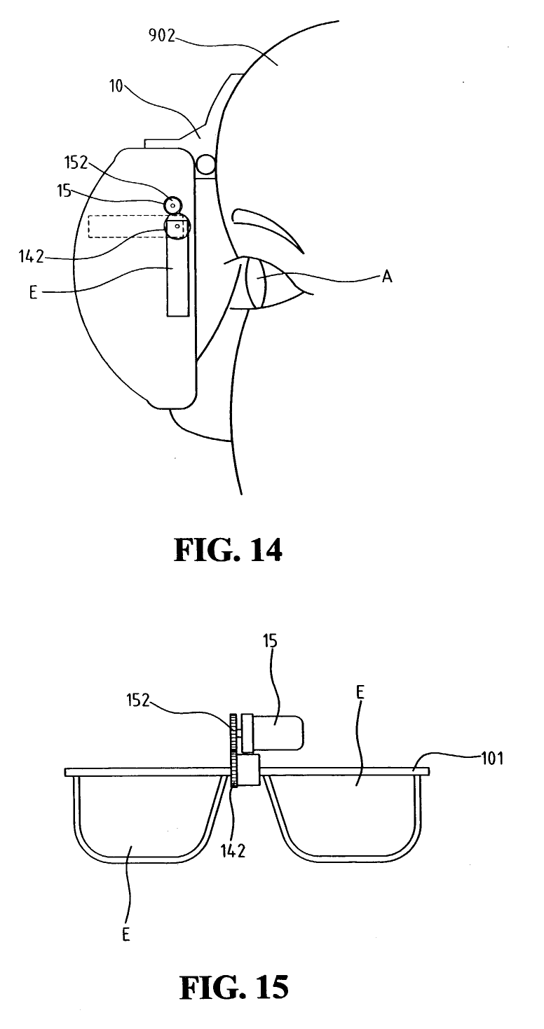

Referring FIGS. 14 and 15, the vision training device constructed in accordance with a fourth embodiment of the present invention is illustrated and generally designate with reference numeral 900D for distinction. The vision training device 900D comprises a fixed frame 10 mountable to the wearer's head 902 and a movable frame 101 mounted to the fixed frame 10 in such a way that the movable frame 101 is rotatable about a horizontal axis substantially parallel to a plane of the wearer's face. Thus, the movable frame 101 is rotatable with respect to the fixed frame 10 between a non-operating position as indicated by phantom lines of FIG. 14 and an operating position as shown in FIG. 15 and the solid lines of FIG. 14. When the movable frame 101 is at the operating position, the movable frame 101 substantially overlaps the fixed frame 10. It is noted that for simplicity, the movable frame 101 is not shown in FIG. 14 while the fixed frame 10 is omitted in FIG. 15.

The movable frame 101 defines two viewing windows (not labeled) substantially corresponding to the eyeballs A of the wearer. A convex-prism lens E is received and retained in each window whereby when the movable frame 101 is at the operating position, the convex-prism lenses E are in the base-in condition for abducting the eyeballs A.

A driving device, such as a step motor 15 controlled by a control circuit (not shown), is mounted to the fixed frame 10 and comprises a driving gear 152A driven gear 142 is mounted to the movable frame 101 and engages the driving gear 152 whereby when the motor 15 is actuated to rotate the driving gear 152 and thus the driven gear 142, the movable frame 101 is rotatable about the horizontal axis between the operating position and the non-operating position.

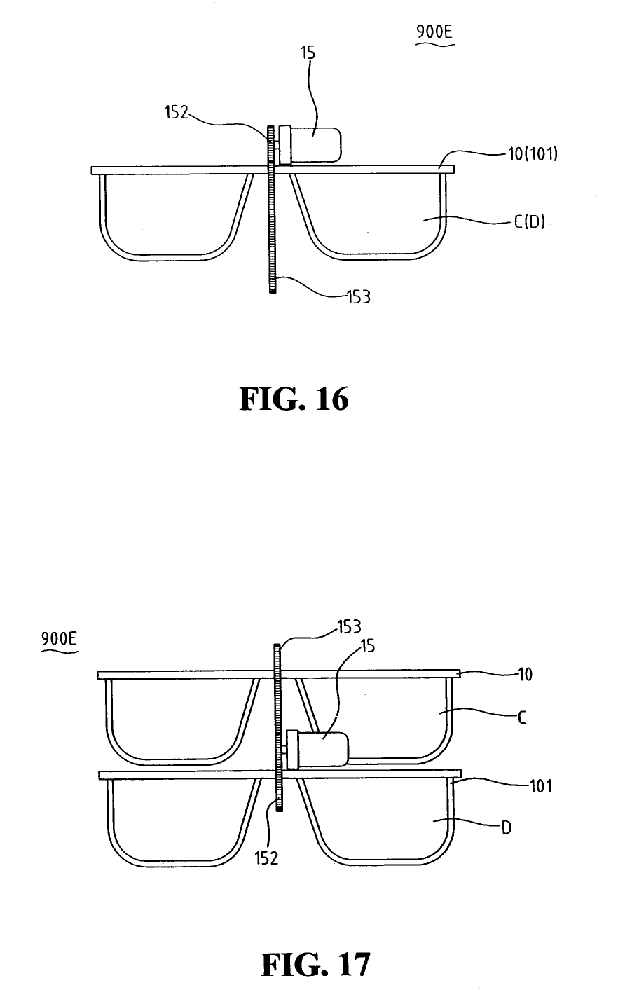

Referring FIGS. 16 and 17, the vision training device constructed in accordance with a fifth embodiment of the present invention is illustrated and generally designate with reference numeral 900E for distinction. The vision training device 900E comprises a fixed frame 10 mountable to the wearer's head (not shown) and a movable frame 101 mounted to the fixed frame 10 in such a way that the movable frame 101 is movable with respect to the fixed frame 10 between an operating position as shown in FIG. 16 where the movable frame 101 substantially overlaps the fixed frame 10 and a non-operating position as shown in FIG. 17 where the movable frame 101 is moved away from the fixed frame 10.

The fixed frame 10 defines two viewing windows (not labeled) substantially corresponding in position to the eyeballs of a wearer and receiving and retaining convex lens D therein whereby the fixed frame 10 and the convex lens D may serve as a pair of eyeglasses for myopia. The movable frame 101 also defines two viewing windows (not labeled) substantially corresponding to the viewing windows of the fixed frame 10 when the movable frame 101 is at the operating position. A prism lens C is received and retained in each window whereby when the movable frame 101 is at the operating position, the prism lenses C are in the base-in condition for abducting the eyeballs.

A driving device, such as a step motor 15 controlled by a control circuit (not shown), is mounted to the fixed frame 10 and comprises a driving gear 152. A vertically-extending rack 153 is mounted to the movable frame 101 and engages the driving gear 152 whereby when the motor 15 is actuated to rotate the driving gear 152 and thus moving the rack 153, the movable frame 101 is moved in the vertical direction with respect to the fixed frame 10 between the operating position and the non-operating position.

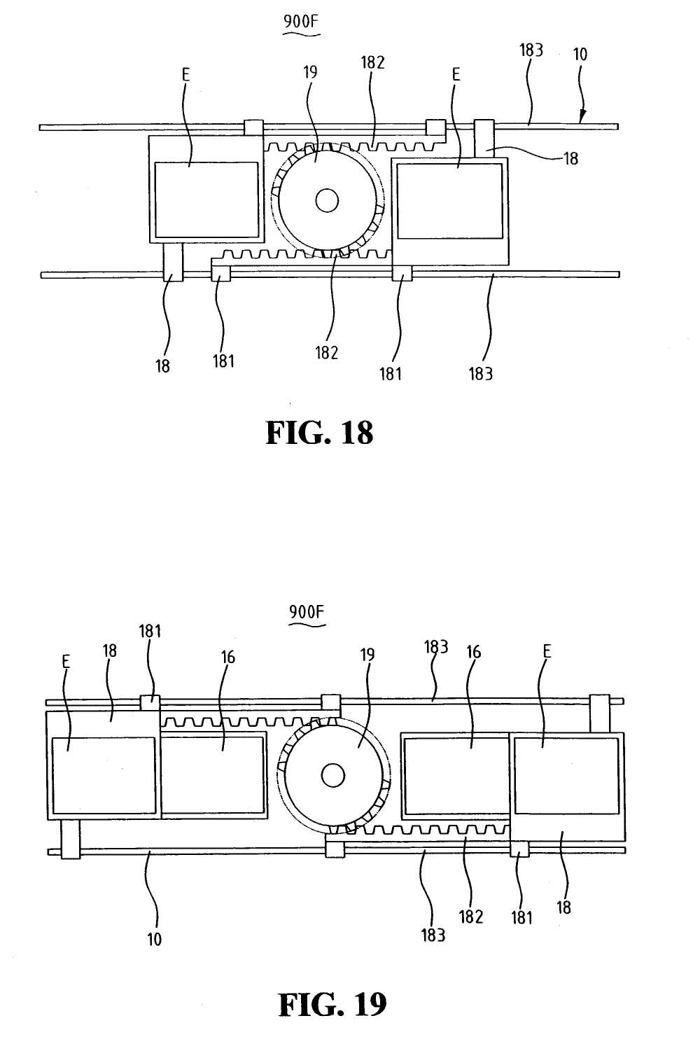

Referring FIGS. 18 and 19, the vision training device constructed in accordance with a sixth embodiment of the present invention is illustrated and generally designate with reference numeral 900F for distinction. The vision training device 900F comprises a fixed frame 10 mountable to the wearer's head (not shown) and defining two viewing windows 16 substantially corresponding in position to a wearer's eyeballs and two movable frames 18 movably mounted to the fixed frame 10 and each defining an opening (not labeled) in which a convex-prism lens E is received and retained. The movable frames 18 are driven by a transmission mechanism to move with respect to the fixed frame 10 between an operating position as shown in FIG. 18 where the convex-prism lenses E of the movable frames 18 substantially correspond in position to the viewing windows 16 of the fixed frame 10 as shown in FIG. 19 where the convex-prism lenses E of the movable frames 101 are off the viewing windows 16.

The transmission mechanism comprises a driving device (not shown) comprising an output gear 19. Each movable frame 18 comprises a rack 183 engaging the output gear 19 whereby when the gear 19 is rotated by the driving device, the rack 183 is caused to drive the movable frames 18 between the operating position and the non-operating position. The fixed frame 10 forms a rail 183 corresponding to each rack 182. In the embodiment illustrated, the rail 183 extends in a horizontal direction and substantially parallel to a plane of the wearer's face. The rack 182 comprises guide blocks 181 movably mounted to the rail 183 and guided thereby for movement along the rail 183. Thus, by means of the driving device, the movable frames 18 are caused to move horizontally along the rails 183 between the operating position and the non-operating position.

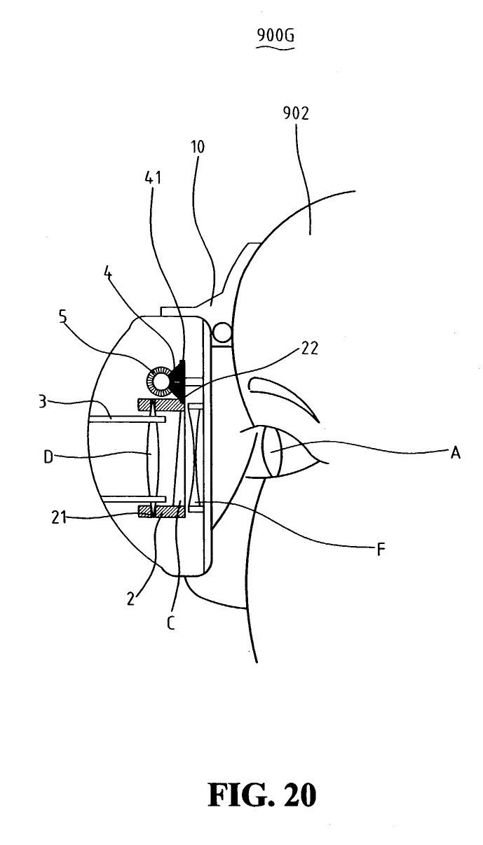

Referring FIG. 20, the vision training device constructed in accordance with a seventh embodiment of the present invention is illustrated and generally designate with reference numeral 900G for distinction. The vision training device 900G comprises a fixed frame 10 mountable to the wearer's head 902 and comprising a concave lens F substantially corresponding in position to each of a wearer's eyeballs A and two lens mounts 2 movably mounted to the fixed frame 10 respectively corresponding to the concave lens F. The lens mounts 2 are driven by a transmission mechanism to rotate with respect to the fixed frame 10 about an axis that extends in a direction substantially normal to the plane of the wearer's face. In other words, the rotation axis is substantially horizontal and extending away from the wearer's eyeball. The mount 2 defines a bore (not labeled) in which a prism lens C is mounted to be rotatable among base-in, base-out and base-down positions. The prism lens C is not allow for linear movement along the axis.

The mount 2 defines a helical groove 21 in an inside surface of the bore thereof A convex lens D has a circumferential edge (not labeled) received in the helical groove 21. Guide posts 3 are fixed on the fixed frame 10 and extend through holes (not labeled) defined in the convex lens D for preventing the convex lens D from rotation. Thus, when the lens mount 2 is rotated, the convex lens D is driven to undergo linear displacement along the axis about which the prism lens C is rotated. A displacement of 1 cm is preferred in the embodiment illustrated.

The transmission mechanism comprises a driving device (not shown) comprising a driving bevel gear 5 mating a driven bevel gear 4 that is rotatably supported on the fixed frame 10. A spur gear 41 is coaxially mounted to the driven bevel gear 4 and mating external teeth 22 of the lens mount 2 for rotating the lens mount 2. Thus, by means of the driving device, the lens mounts 2 are caused to rotate about the axis which in turn induces rotation of the prism lens C about the axis, while driving the convex lens D to move horizontally along the guide posts 3.

In the seventh embodiment, the power of the convex lens D is approximately +10 Diopter−+13 Diopter, the power of the prism lens C is approximately 4 ΔDiopter−8 ΔDiopter, and the power of the concave lens F is approximately −10 Diopter−−13 Diopter.

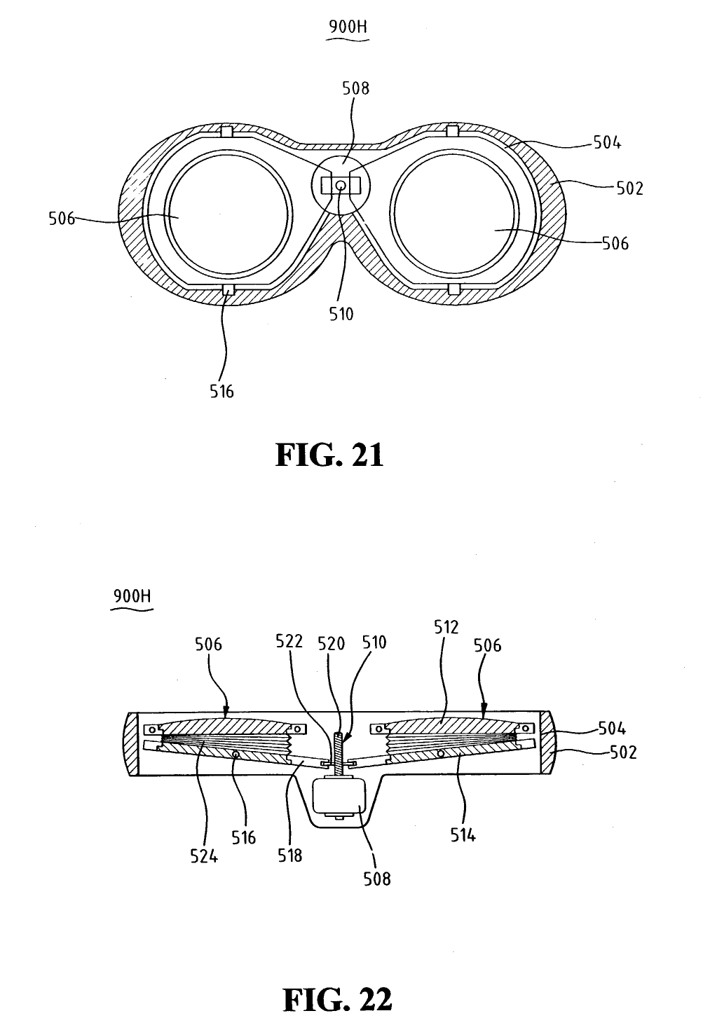

Referring to FIGS. 21 and 22, the vision training device constructed in accordance with an eighth embodiment of the present invention is shown, which is designated with reference numeral 900H, comprising a fixed frame 502, which can be embodied as an eyeglass frame for wearing on a wearer's head. The frame 502 defines two viewing windows 504 substantially corresponding in position to the eyeballs of the wearer. An adjustable prism lens assembly 506 is mounted in each window 504. A driving device 508, comprising a motor controlled by a control circuit, is mounted to the frame 502 for operating the adjustable prism lens assembly 506. In the embodiment illustrated, the driving device 508 is arranged between the adjustable prism lens assemblies 506. However, the driving device 508 can be mounted to any suitable position on the fixed frame 502.

The driving device 508 comprises a transmission mechanism 510 coupled to the adjustable prism lens assemblies 506 for operating the adjustable prism lens assemblies 506. The operation of the adjustable prism lens assemblies 506 change the power of the prism lens assemblies 506 thereby changing the refraction of the light passing through the adjustable prism lens assemblies 506. The function of the prism lens assemblies 506 in the embodiment is to refract the light passing therethrough. In other words, light incident onto the wearer's eyeballs is changed from a first state of refraction to a second state of refraction for repeatedly abducting the eyeballs and thus realizing the training of vision in accordance with the present invention.

The adjustable prism lens assembly 506 comprises a first lens 512, such as a convex lens, and a second lens 514, such as a flat lens. The first lens 512 is fixed to the fixed frame 502, while the second lens 514 is movably retained in the viewing window 504 of the fixed frame 502. The driving device 508 is coupled to the second lens 514 by the transmission mechanism 510 for moving the second lens 514 and thus changing the spatial relationship between the first and second lenses 512 and 514. In the embodiment illustrated, the second lens 514 is pivoted to the fixed frame 502 whereby the second lens 514 is rotatable about a pivot 516 extending in a vertical direction. The second lens 514 has an inner edge 518, that is the edge facing the other second lens, connected to the transmission mechanism 510 whereby the second lens 514 is rotatable about the pivot 516 with respect to the first lens 512 and the included angle therebetween is changed from a first angle corresponding to the first refraction state to a second angle corresponding to the second refraction state.

The transmission mechanism 510 comprises a threaded rod 520 connected to the driving device 508. An inner-threaded moveable member 522, to which the inner edge 518 of each second lens 514 is attached, threadingly engages the rod 520 to undergo linear displacement along the rod 520 when the rod 520 is rotated. Thus with the rotation of the rod 520 by the driving device 508, the movable member 522 moves along the rod 520 and change the included angle between the first and second lens 512, 514 in a stepless manner. Thus, the refraction of the light passing through the prism lens assembly 506 can accordingly be changed in a stepless manner.

Preferably, a flexible tube 524, such as a bellow tube, is connected between the first and second lenses 512, 514 to define a hermetic interior space between the first and second lenses 512, 514. A fluid having a desired refraction index is filled in the interior space for optimizing the refraction result of the prism lens assembly 506. To avoid unnecessary change of inside pressure of the fluid, the total volume of the interior space is maintained substantially constant. This can be done by for example arranging the pivot 516 at substantially center of the second lens 514. Alternatively, a fluid reservoir (not shown) can be provided and in fluid communication with the interior space to take the change of inside pressure of the fluid.

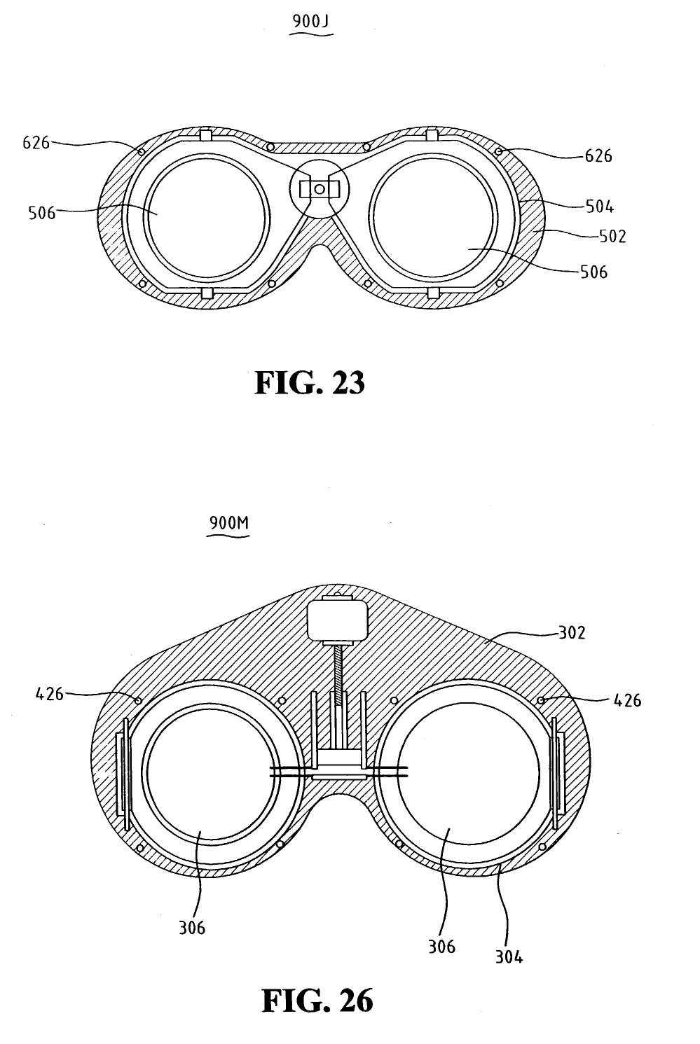

Also referring to FIG. 23, the vision training device constructed in accordance with a ninth embodiment of the present invention is illustrated, which is generally designated with reference numeral 900J. The vision training device 900J is a modification of the vision training device 900H, comprising a fixed frame 502 defining two viewing windows 504 in which adjustable prism lens assemblies 506 are mounted. A plurality of illuminating elements 626, such as light emitting diodes, are mounted to the frame 502 along a circumferential edge of each window 504. The illuminating elements 626 can be lighted in accordance with a predetermined sequence to attract and thus move the eyeballs of the wearer. The lighting of the illuminating elements 626 can be done corresponding to the adjustment of the second lens 514.

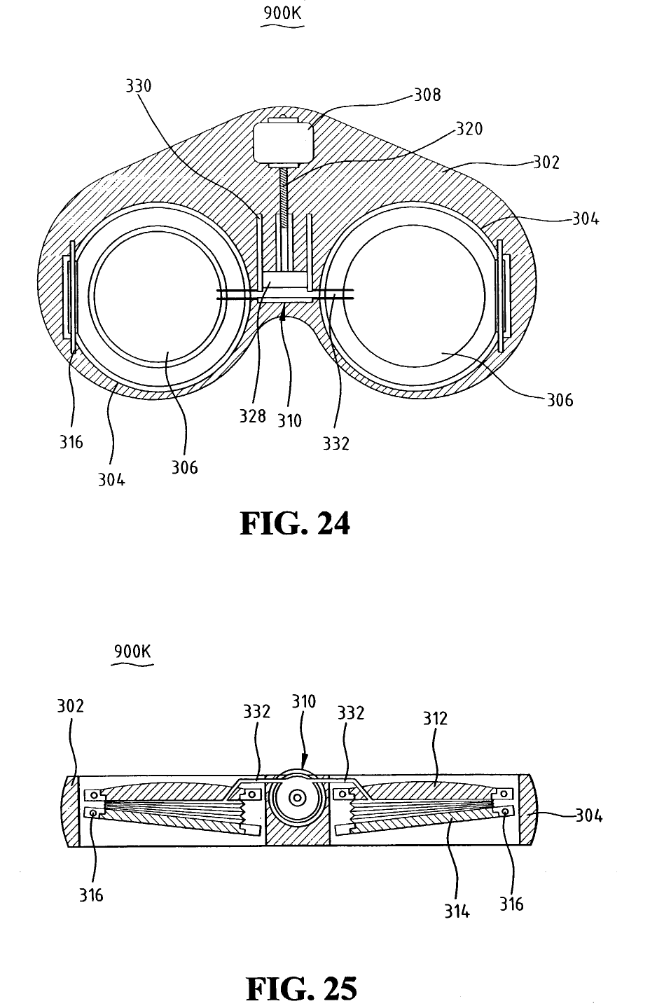

Referring to FIGS. 24 and 25, the vision training device constructed in accordance with a tenth embodiment of the present invention is shown, which is designated with reference numeral 900K, comprising a fixed frame 302, which can be embodied as an eyeglass frame for wearing on a wearer's head. The frame 302 defines two viewing windows 304 substantially corresponding in position to the eyeballs of the wearer. An adjustable prism lens assembly 306 is mounted in each window 304. A driving device 308, comprising a motor controlled by a control circuit, is mounted to the frame 302 for operating the adjustable prism lens assembly 306. In the embodiment illustrated, the driving device 308 is arranged between the adjustable prism lens assemblies 306. However, the driving device 308 can be mounted to any suitable position on the fixed frame 302.

The driving device 308 comprises a transmission mechanism 310 coupled to the adjustable prism lens assemblies 306 for operating the adjustable prism lens assemblies 306. The operation of the adjustable prism lens assemblies 306 changes the power of the prism lens assemblies 306 thereby changing the refraction of the light passing through the adjustable prism lens assemblies 306. The function of the prism lens assemblies 306 in the embodiment is to refract the light passing therethrough. In other words, light incident onto the wearer's eyeballs is changed from a first state of refraction to a second state of refraction for repeatedly abducting the eyeballs and thus realizing the training of vision in accordance with the present invention.

The adjustable prism lens assembly 306 comprises a first lens 312 and a second lens 314. The first lens 312 is fixed to the fixed frame 302, while the second lens 314 is movably retained in the viewing window 304 of the fixed frame 302. The driving device 308 is coupled to the second lens 314 by the transmission mechanism 310 for moving the second lens 314 and thus changing the spatial relationship between the first and second lenses 312 and 314. In the embodiment illustrated, the second lens 314 is pivoted to the fixed frame 302 whereby the second lens 314 is rotatable about a pivot 316 extending in a vertical direction and an included angle between the first and second lenses 312, 314 is changed from a first angle corresponding to the first refraction state to a second angle corresponding to the second refraction state.

The transmission mechanism 310 comprises a threaded rod 320 connected to the driving device 308. A piston forms an inner-threaded bore (not labeled) threadingly engaging the threaded rod 320 for undergoing linear displacement inside a cylinder 330 when the rod 320 is rotated by the driving device 308.

A flexible tube 324, such as a bellow tube, is connected between the first and second lenses 312, 314 to define a hermetic interior space between the first and second lenses 312, 314, which is in fluid communication with the cylinder 330 via a conduit 332. A fluid having a desired refraction index is filled in the cylinder 330 and the interior space between the first and second lenses 312, 314. The fluid is driven into and drawn out of the interior space under the linear displacement of the piston 328 inside the cylinder 330. Thus, the second lens 314 is forced to rotate about the pivot 316 with respect to the first lens 312, changing the included angle between the first and second lens 312, 314 in a stepless manner. Thus, the refraction of the light passing through the prism lens assembly 306 can accordingly be changed in a stepless manner. In this way, the cylinder 330 serves as a fluid supply source for the interior space between the first and second lenses 312, 314.

Also referring to FIG. 26, the vision training device constructed in accordance with an eleventh embodiment of the present invention is illustrated, which is generally designated with reference numeral 900M. The vision training device 900M is a modification of the vision training device 900K, comprising a fixed frame 302 defining two viewing windows 304 in which adjustable prism lens assemblies 306 are mounted. A plurality of illuminating elements 426, such as light emitting diodes, are mounted to the frame 302 along a circumferential edge of each window 304. The illuminating elements 426 can be lighted in accordance with a predetermined sequence to attract and thus move the eyeballs of the wearer. The lighting of the illuminating elements 426 can be done corresponding to the adjustment of the second lens 314.

Although the present invention has been described with reference to the preferred embodiments thereof, it is apparent to those skilled in the art that a variety of modifications and changes may be made without departing from the scope of the present invention which is intended to be defined by the appended claims.

What is claimed is

1. A vision training device comprising:
a fixed frame adapted to be positioned in front of a wearer's face, the fixed frame defining at least a window corresponding in position to an eye of the wearer, through which light passes;
an optic system comprising a prism lens movably mounted to the fixed frame to be movable between first and second positions, wherein in the first position, light is allowed to pass in a first state and in the second position, light is allowed to pass in a second state that is different from the first state; and
a transmission system coupled to and selectively driving the prism lens between the first and second positions.

2. The vision training device as claimed in claim 1, wherein the optic system further comprises a convex lens covering the window.

3. The vision training device as claimed in claim 2, wherein the convex lens is fixedly attached to the window.

4. The vision training device as claimed in claim 2, wherein the convex lens is linearly displaceable along an axis to change a distance between the convex lens and the prism lens.

5. The vision training device as claimed in claim 4, wherein the linear displacement of the convex lens is within a range of 1 cm.

6. The vision training device as claimed in claim 4, wherein the optic system further comprises a concave lens mounted to the fixed frame and located between the prism lens and the wearer's eye.

7. The vision training device as claimed in claim 6, wherein the displacement of the convex lens is within a range of 1 cm.

8. The vision training device as claimed in claim 4, wherein the transmission system comprises a rotatable lens mount rotatably mounted to the fixed frame, the lens mounted defining a bore in which the prism lens is mounted to be rotatable with the mount, a helical groove being defined in an inside surface of the bore for receiving an edge of the convex lens whereby the rotation of the lens mount causes the convex lens to undergo linear displacement, guide posts extending from the fixed frame and extending through holes defined in the convex lens for preventing rotation of the convex lens and for guiding the linear displacement of the convex lens.

9. The vision training device as claimed in claim 1, wherein the optic system comprising a convex-prism lens comprising a convex configuration formed on a prism lens, the convex-prism lens having a base that has a great thickness.

10. The vision training device as claimed in claim 9, wherein the transmission system comprises a rotatable ring in which the convex-prism lens is mounted, a plurality of rollers being rotatably mounted to the fixed frame and engaging an outer circumference of the ring to rotatably support the ring.

11. The vision training device as claimed in claim 10, wherein the transmission system further comprises a gear system engaging external teeth formed on the outer circumference of the ring and driven by a driving device to rotate the ring whereby the convex-prism lens is moved between a first position and a second position.

12. The vision training device as claimed in claim 11, wherein the fixed frame defines two viewing windows respectively corresponding to the eyes of the wearer and two rotatable rings arranged in correspondence with the two windows, each ring receiving and retaining a convex-prism lens, both rings engaged by the gear system to be rotated thereby simultaneously for moving the convex-prism lenses between the second position where the bases are close to each other and the first position where the bases are away from each other.

13. The vision training device as claimed in claim 12, wherein the gear system comprises a plurality of first gears engaging each other and engaging the teeth of the rings, the gear system further comprising a pair of mated bevel gears of which a first one is driven by a driving device and a second one mounted to one of the first gears.

14. The vision training device as claimed in claim 1, wherein the transmission system comprises a rack mounted to the prism lens and a gear mating the rack and driven by a driving device whereby the prism lens is moved by the rack between the first and second positions.

15. The vision training device as claimed in claim 14, wherein the fixed frame defines two viewing windows respectively corresponding to the eyes of the wearer, two prism lenses being arranged in correspondence to the windows, the transmission system comprising two racks respectively mounted to the prism lenses and a gear mating the racks and driven by a driving device to move the prism lens between the first and second positions via the racks in a linear horizontal direction.

16. The vision training device as claimed in claim 14, wherein the fixed frame defines two viewing windows respectively corresponding to the eyes of the wearer, a movable frame to which two prism lenses are mounted in correspondence to the windows, the transmission system comprising a rack mounted to the movable frame for moving the prism lens between the first and second positions via the rack in a linear vertical direction.

17. The vision training device as claimed in claim 1, wherein the transmission system comprises a rotatable ring carrying the prism lens, the ring being rotatable about a pivot to move the prism lens between the first position where the prism lens does not overlap the window and the second position where the prism overlaps the window.

18. The vision training device as claimed in claim 17, wherein the pivot extends in a direction substantially normal to the wearer's face.

19. The vision training device as claimed in claim 18, wherein the transmission system comprises a gear system coupled between a driving device and the pivot for rotating the pivot.

20. The vision training device as claimed in claim 19, wherein the pivot extends in a direction substantially normal to the wearer's face.

21. The vision training device as claimed in claim 1, wherein the transmission system repeatedly and cyclically moves the prism lens between the first and second positions.

22. The vision training device as claimed in claim 21, wherein the prism lens is staying in the first position for a period of 5-20 seconds, while in the second position for a period of 10-30 seconds.

23. The vision training device as claimed in claim 1, wherein the frame is adapted to be mounted to the head of the wearer.

24. The vision training device as claimed in claim 1, wherein the frame is made in the form of a pair of eyeglasses.

25. The vision training device as claimed in claim 1, wherein the frame is adapted to be positioned on a fixture in front of the wearer's eyes.

26. A method for training vision comprising the following steps:
(1) providing a prism lens having a thick base, the prism lens being placed at a first position in front of an eye of a person to allow light to get incident onto the eye in a first state for a first period of time;
(2) manipulating the prism lens to have the base moved to a second position that is different from the first position, which changes the incident light from the first state to a second state for a second period of time;
(3) manipulating the prism lens to move the base back to the first position and change the incident light from the second state back to the first state; and
(4) repeating steps (2) and (3).

27. The method as claimed in claim 26, wherein the second state of the prism lens causes the abduction of the eye, while the first state causes adduction of the eye.

28. The method as claimed in claim 26, wherein the first state of the prism lens causes upward turning of the eye.

29. The method as claimed in claim 26, wherein the first period of time is approximately 5-20 seconds, while the second period of time is approximately 10-30 seconds.

30. The method as claimed in claim 26, wherein the prism lens is integrated with a convex lens to form a convex-prism lens.

31. A vision training device comprising:
a fixed frame adapted to be positioned in front of a wearer's face, the fixed frame defining at least a window corresponding in position to an eye of the wearer, through which light passes; and
an adjustable optic system movably mounted to the fixed frame, comprising:
a first lens mounted to the window, and
a second lens mounted to the window and facing the first lens forming a first included angle therebetween to allow light to pass therethrough in a first state, the second lens being movable with respect to the first lens to change the first included angle to a second, different included angle for changing the light from the first state to a second state.

32. The vision training device as claimed in claim 31 further comprising a driving device coupled to the second lens by a transmission mechanism for moving the second lens with respect to the first lens.

33. The vision training device as claimed in claim 31 further comprising a flexible tube connected between the first and second lenses and defining a hermetic interior space between the first and second lenses in which a light transmitting fluid is filled.

34. The vision training device as claimed in claim 31, wherein the second lens is pivoted to the window for being rotatable with respect to the first lens to change the first included angle to the second included angle.

35. The vision training device as claimed in claim 31 further comprising a plurality of illuminating elements mounted along a circumference of the window to be selectively illuminated to attract the eyeballs of the wearer.

36. The vision training device as claimed in claim 31, wherein the first lens comprises a convex lens.

37. The vision training device as claimed in claim 32 further comprising a flexible tube connected between the first and second lenses and defining a hermetic interior space between the first and second lenses in which a light transmitting fluid is filled.

38. The vision training device as claimed in claim 32, wherein the second lens is pivoted to the window for being rotatable with respect to the first lens to change the first included angle to the second included angle.

39. The vision training device as claimed in claim 38, wherein the second lens is pivoted to the window with a central portion thereof, the transmission mechanism comprising a linearly movable member connected to an edge of the second lens for rotating the second lens about the pivot when the linearly movable member is linearly moved.

40. The vision training device as claimed in claim 39, wherein the transmission mechanism comprises a motor and a threaded rod connected to the motor, the linearly movable member defining an inner-threaded bore threadingly engaging the threaded rod for being linearly moved along the rod by the rotation of the rod caused by the motor.

41. The vision training device as claimed in claim 40 further comprising a plurality of illuminating elements mounted along a circumference of the window to be selectively illuminated to attract the eyeballs of the wearer.

42. The vision training device as claimed in claim 40, wherein the first lens comprises a convex lens.

43. The vision training device as claimed in claim 37, wherein the second lens is pivoted to the window for being rotatable with respect to the first lens to change the first included angle to the second included angle.

44. The vision training device as claimed in claim 43, wherein the second lens is pivoted to the window with a central portion thereof, the transmission mechanism comprising a linearly movable member connected to an edge of the second lens for rotating the second lens about the pivot when the linearly movable member is linearly moved.

45. The vision training device as claimed in claim 44, wherein the transmission mechanism comprises a motor and a threaded rod connected to the motor, the linearly movable member defining an inner-threaded bore threadingly engaging the threaded rod for being linearly moved along the rod by the rotation of the rod caused by the motor.

46. The vision training device as claimed in claim 45 further comprising a plurality of illuminating elements mounted along a circumference of the window to be selectively illuminated to attract the eyeballs of the wearer.

47. The vision training device as claimed in claim 45, wherein the first lens comprises a convex lens.

48. The vision training device as claimed in claim 37, wherein the second lens is pivoted to the window with an edge thereof, the transmission mechanism comprising a fluid supply source for filling fluid into the interior space defined by the flexible tube to force the second lens to rotate about the pivot and thus changing the first included angle to the second included angle.

49. The vision training device as claimed in claim 48, wherein the fluid supply source comprises a cylinder in which the fluid is filled and in fluid communication with the interior space of the flexible tube by a conduit, a piston driven by the driving device to move in the cylinder for driving the fluid into the interior space of the flexible tube.

50. The vision training device as claimed in claim 49, wherein the driving device comprises a motor and a threaded rod connected to the motor, the piston being threadedly coupled to the threaded rod whereby when the rod is rotated by the motor, the piston is linearly moved in the cylinder.

51. The vision training device as claimed in claim 50 further comprising a plurality of illuminating elements mounted along a circumference of the window to be selectively illuminated to attract the eyeballs of the wearer.

52. The vision training device as claimed in claim 50, wherein the first lens comprises a convex lens.
